(19) United States
(12) Patent Application Publication
(10) Pub. No.: US 2013/0065258 A1
(43) Pub. Date: Mar. 14, 2013

(54) COLORECTAL CANCER MARKER GALECTIN, METHOD FOR ANALYZING GALECTIN CONCENTRATION IN BLOOD SAMPLE, AND KIT FOR DETECTING COLORECTAL CANCER MARKER GALECTIN

(71) Applicants: Makoto Watanabe, Osaka-shi (JP); Ei-ichi Matsuo, Kyoto-shi (JP); Naoki Kaneko, Kyoto-shi (JP); Toshiya Matsubara, Nishinomiya-shi (JP); Osamu Nishimura, Kawanishi-shi (JP); Masaki Mori, Suita-shi (JP); Ichiro Takemasa, Toyonaka-shi (JP)

(72) Inventors: Makoto Watanabe, Osaka-shi (JP); Ei-ichi Matsuo, Kyoto-shi (JP); Naoki Kaneko, Kyoto-shi (JP); Toshiya Matsubara, Nishinomiya-shi (JP); Osamu Nishimura, Kawanishi-shi (JP); Masaki Mori, Suita-shi (JP); Ichiro Takemasa, Toyonaka-shi (JP)

(21) Appl. No.: 13/699,592

(22) PCT Filed: Jan. 28, 2011
(86) PCT No.: PCT/JP2011/051760

§ 371 Date: Nov. 30, 2012

(30) Foreign Application Priority Data
May 25, 2010 (JP) 2010-119420

(51) Int. Cl. G01N 33/574; C07K 14/47; G01N 21/64

(52) U.S. Cl. 435/7.92; 436/501; 530/396

(57) ABSTRACT

The present invention provides a tumor screening marker that can be actually used in clinical practice to detect colorectal cancer, and a tumor progression marker that can complement CEA or CA19-9. Galectin-1 used as a tumor screening marker or a tumor progression marker for colorectal cancer. Galectin-3 used as a tumor screening marker. Galectin-4 used as a tumor progression marker, a tumor screening marker, or a prognostic prediction marker for colorectal cancer. A method of analyzing the galectin concentration in a collected blood sample using the galectin. A colorectal cancer marker detection kit comprising a detection antibody selected from the group consisting of a fluorescently labeled galectin-1 antibody, a fluorescently labeled galectin-3 antibody, and a fluorescently labeled galectin-4 antibody.

### TECHNICAL FIELD

The present invention relates to a colorectal cancer marker galectin, a method of analyzing a galectin concentration in a collected blood sample, and a kit for detecting a colorectal cancer marker galectin. The present invention relates to a field of clinical diagnosis such as diagnosis and prognostication of colorectal cancer.

### BACKGROUND ART

As one of tools for diagnosis, examination, and follow-up of colorectal cancer (CRC), a blood test may be performed. A blood test makes it possible to detect cancer, estimate the extent of cancer, or determine the prognosis of cancer by measuring the concentration of a certain protein (cancer marker) present in the blood of a patient. Such colorectal cancer markers are described in, for example, Anticancer Research, 2004, 24(4), 2519-2530 (Non-Patent Document 1).

Examples of current typical colorectal cancer markers include carcinoembryonic antigen (CEA) and carbohydrate antigen 19-9 (CA19-9). Both these markers show a low positive rate especially in the early stage of cancer, and are therefore not suitable as “tumor screening markers”. However, these markers deliver excellent performance as “tumor progression markers” for use in, for example, follow-up after surgery, and the use of these markers for colorectal cancer patients is covered by health insurance in Japan.

American Society of Clinical Oncology (ASCO) recommends the use of CEA, not as a tumor screening marker, but as a “tumor progression marker” for prognostication, staging, and drug efficacy evaluation. On the other hand, ASCO has concluded that CA19-9 is not suitable for use alone as a colorectal cancer marker because current data is insufficient to support the use of CA19-9 as a colorectal cancer marker.

U.S. FDA also approves the use of CEA as a colorectal cancer marker.

As described above, CEA and CA19-9 are used around the world including Japan and USA as “tumor progression markers”. This is because the levels of these markers in a colorectal cancer patient accurately reflect the disease state of cancer in the body of the patient (in the case of colorectal cancer, the disease state of cancer may be represented by, for example, the difference in the stage of cancer progression determined by the total amount of cancer present in the body or the extent of metastasis). That is, in almost all the cases of colorectal cancer patients whose levels of these markers measured with a blood test exceeded threshold values, the marker levels are significantly reduced after surgery (i.e., are returned to the threshold values or less) but are increased (i.e., exceed the threshold values) if a metastasis or relapse occurs. This is utilized to allow colorectal cancer to be monitored by measuring the blood levels of these markers.

Galectins are lectins that specifically recognize p-linked galactose, and are known to control differentiation or growth of cells and apoptosis as well as to play a role in signal transmission. JP 2008-14937 A (Patent Document 1) reports that higher expression of galectin has been detected in cancerous parts than in non-cancerous parts of colorectal tissues.

### ART DOCUMENT PRIOR TO THE APPLICATION

### Patent Document

Patent Document 1: JP 2008-14937 A

### Non-Patent Document

Non-Patent Document 1: Anticancer Research, 2004, vol. 24, no. 4, p. 2519-2530

### DISCLOSURE OF THE INVENTION

### Problem to be Solved by the Invention

It is said that the ratio of colorectal cancer patients whose concentration of CEA or CA19-9 in a blood sample exceeds a threshold value and who can undergo cancer monitoring using such a marker is 30 to 60% (CEA) or 11 to 34% (CA19-9) of the total at most. As described above, CEA or CA19-9 is practically used as a “tumor progression marker”, but it is often the case that some colorectal cancer patients are not positive for these markers. Therefore, in order to achieve more exhaustive monitoring of disease state, there is a strong demand in clinical practice for novel markers applicable to many patients not covered by CEA and CA19-9.

Further, it is also known that there is a case where the level of CEA or CA19-9 varies with factors other than cancer. Therefore, in order to achieve more accurate monitoring of disease state, there is a strong demand in clinical practice for novel markers that can complement CEA or CA19-9 used as a marker.

Further, there are no “tumor screening markers” used in a blood test to easily determine the presence or absence of colorectal cancer.

For the above reasons, development of “tumor screening markers” for colorectal cancer detection and development of “tumor progression markers” that can complement CEA or CA19-9 are needed urgently.

It is to be noted that the effectiveness of measurement of a galectin concentration in a collected blood sample for detection of a colorectal cancer patient has not been demonstrated at all. Therefore, there has been hitherto no suggestion of the possibility that the presence or absence of colorectal cancer can be easily and effectively determined by measuring galectin with a blood test.

An object of the present invention is to provide a “tumor screening marker” that can be actually used in clinical practice to detect colorectal cancer, and a “tumor progression marker” that can complement CEA or CA19-9. Another object of the present invention is to provide a method of analyzing a collected blood sample using such a marker.

### Means for Solving the Problem

The present inventors have intensively studied, and as a result, have found the effectiveness of measurement of galectin in a collected blood sample, the effectiveness of galectin-4 as a tumor progression marker, a tumor screening marker, and a prognostic prediction marker, the effectiveness of galectin-1 as a tumor screening marker and a tumor progression marker, and the effectiveness of galectin-3 as a tumor screening marker, which has led to the completion of the present invention.

The following is directed to a novel colorectal cancer marker.

It is to be noted that in the present invention, the “tumor progression marker” refers to a tumor marker whose concentration increases as the disease state of cancer progresses. The tumor progression marker may be used when the presence of cancer has already been confirmed for the purpose of determining the extent of the cancer or monitoring the disease state of the cancer.

In the present invention, the “tumor screening marker” refers to a tumor marker whose concentration is higher when cancer is present than when cancer is not present. The tumor screening marker may be used when the presence of cancer in the body has not yet been confirmed for the purpose of determining whether cancer is present or not. Among the tumor screening markers, one whose blood concentration increases in the early stage of cancer is preferred in that it is suitable for early diagnosis.

In the present invention, the “prognostic prediction marker” refers to a marker used to predict disease prognosis (e.g., after 5 years of initiation of treatment) at some point in time (e.g., at the initiation of treatment).

In this specification, Sₙ refers to a collected blood sample derived from blood collected at some point in time Tₙ, Cₙ refers to a measured value of a colorectal cancer marker acquired from the sample Sₙ, C_{ref} refers to a reference value of the colorectal cancer marker, and Pₙ refers to the step of acquiring the measured value Cₙ from the sample Sₙ and comparing the measured value Cₙ with the reference value C_{ref}. Further, Cₜₕ refers to a threshold value of the colorectal cancer marker. It is to be noted that in this specification, the term “positive rate” refers to the ratio (%) of patients whose measured value of the colorectal cancer marker is higher than Cₜₕ (i.e., who are positive for the colorectal cancer marker) to the total patients as analysis objects.

It is to be noted that, when the colorectal cancer marker is, for example, galectin-1, a measured value of galectin-1 is referred to as Cₙ[G1], a reference value of galectin-1 is referred to as C_{ref}[G1], and a threshold value of galectin-1 is referred to as Cₜₕ[G1]. Similarly, a measured value of galectin-3 is referred to as Cₙ[G3], a reference value of galectin-3 is referred to as C_{ref}[G3], and a threshold value of galectin-3 is referred to as Cₜₕ[G3], and a measured value of galectin-4 is referred to as Cₙ[G4], a reference value of galectin-4 is referred to as C_{ref}[G4], and a threshold value of galectin-4 is referred to as Cₜₕ[G4].

Further, when the colorectal cancer marker is, for example, galectin-3 or galectin 4, there is a case where the measured value of galectin-3 Cₙ[G3] or the measured value of galectin-4 Cₙ[G4] is referred to as Cₙ[G3/G4]. The same goes for the other values.

In this specification, when the term “galectins” is simply used without referring to the kind of galectins (i.e., galectin-1, galectin-3, and galectin-4), the term “galectins” is used as a generic name for galectin-1, galectin-3, and galectin-4.

(1) A colorectal cancer marker selected from the group consisting of galectin-3 and galectin-4.

(2) The colorectal cancer marker according to (1), wherein the galectin-4 is used as a tumor progression marker, a tumor screening marker, or a prognostic prediction marker.

(3) The colorectal cancer marker according to (1), wherein the galectin-3 is used as a tumor screening marker.

The following is directed to a method of analyzing a galectin concentration in a collected blood sample. The analysis method according to the present invention comprises a comparison between a measured value of galectin in a collected blood sample and a reference value of galectin.

In the present invention, the reference value of the colorectal cancer marker includes a measured value of the colorectal cancer marker acquired from another collected blood sample, and a threshold value specific to each of the colorectal cancer markers.

(4) A method of analyzing a galectin concentration in a collected blood sample, the method comprising the step Pₙ of measuring a concentration of a colorectal cancer marker selected from the group consisting of galectin-3 and galectin-4 in a collected blood sample Sₙ derived from an individual to acquire a measured value Cₙ[G3/G4] and comparing the measured value Cₙ[G3/G4] with a reference value C_{ref}[G3/G4] of the colorectal cancer marker, thereby analyzing the galectin concentration.

The following is directed to one embodiment of a method using galectin-4 as a “tumor progression marker”. This embodiment comprises a comparison between a measured value of galectin in a collected blood sample and a measured value of galectin in a blood sample previously collected and/or a threshold value of galectin.

(5) The method according to (4), further comprising, prior to the step Pₙ (n≧1), the step Pₙ₋₁ of measuring a concentration of galectin-4 in a collected blood sample Sₙ₋₁ derived from the same individual and collected before collection of the blood sample Sₙ to acquire a measured value Cₙ₋₁[G4], wherein

the reference value C_{ref}[G4] compared with the measured value Cₙ[G4] in the step Pₙ is selected from the group consisting of the measured value Cₙ₋₁[G4] and a threshold value Cₜₕ[G4] of galectin-4.

In the above (5), the individual may be one that has undergone treatment for colorectal cancer before the step Pₙ.

One example of the embodiment according to the above (5) is schematically shown in FIG. 1.

The following is directed to an embodiment of the method using galectin-4 as a “tumor progression marker”, in which the blood sample is derived from an individual that has been treated by at least surgery. This embodiment is applied to monitor an individual that has been confirmed to have no residual primary lesion of colorectal cancer after surgery (i.e., curability is A or B), and requires that a measured value of galectin in a blood sample collected before treatment for colorectal cancer exceeded a threshold value and a measured value of galectin in a blood sample collected after the treatment was below the threshold value. When such a requirement is satisfied, a measured value of galectin in a blood sample further collected thereafter is compared with the threshold value. One example of this embodiment is schematically shown in FIG. 2.

(6) The method according to (5), comprising, prior to the step Pₙ (n≧2), the step P₁ of measuring a concentration of galectin-4 in a collected blood sample S₁ derived from the same individual and collected before collection of the blood sample Sₙ to acquire a measured value C₁[G4], and the step P₀ of measuring a concentration of galectin-4 in a collected blood sample S₀ derived from the same individual and collected before collection of the blood sample S₁ to acquire a measured value C₀[G4], wherein

the individual has undergone surgery for colorectal cancer between the step P₀ and the step P₁, the measured value C₀[G4] acquired in the step P₀ exceeds the threshold value Cₜₕ[G4] of galectin-4, and the measured value C₁[G4] acquired in the step P₁ is below the threshold value Cₜₕ[G4], and the reference value C_{ref}[G4] compared with the measured value Cₙ[G4] in the step Pₙ is the threshold value Cₜₕ[G4].

(7) The method according to (6), wherein the individual has further undergone non-surgical therapy (e.g., radiation therapy or chemotherapy) for colorectal cancer between the step P₁ and the step Pₙ.

The following are directed to embodiments of the method using galectin-4 as a “tumor progression marker”, in which the blood sample is derived from an individual that has been treated by at least non-surgical therapy (e.g., radiation therapy or chemotherapy). In the following, the phrase “has undergone at least non-surgical therapy for colorectal cancer” includes both cases where the individual has undergone only non-surgical therapy, and where the individual has undergone surgical therapy before non-surgical therapy.

Further, the following embodiment requires that the non-surgical therapy is performed once, and that a measured value (Cₙ₋₁) of galectin in a blood sample collected before treatment for colorectal cancer with the non-surgical therapy (Tₙ₋₁) exceeded a threshold value (in a case where surgical therapy has been performed before the non-surgical therapy, it is required that the measured value (Cₙ₋₁) of galectin still exceeded the threshold value after the surgical therapy (Tₙ₋₁)). When such a requirement is satisfied, a measured value (Cₙ) of galectin in a blood sample further collected thereafter (Tₙ) is compared with the measured value (Cₙ₋₁) and the threshold value (Cₜₕ).

(8) The method according to (5), wherein the individual has undergone at least non-surgical therapy for colorectal cancer between the step Pₙ₋₁ and the step Pₙ,

the measured value Cₙ₋₁[G4] acquired in the step Pₙ₋₁ exceeds the threshold value Cₜₕ[G4] of galectin-4, and the reference value C_{ref}[G4] compared with the measured value Cₙ[G4] in the step Pₙ is the threshold value Cₜₕ[G4] and the measured value Cₙ₋₁[G4].

On the other hand, the following embodiment requires that the non-surgical therapy is performed two or more times, and that a measured value of galectin in a blood sample collected before treatment for colorectal cancer with the non-surgical therapy (T₀) exceeded a threshold value (in a case where surgical therapy has been performed before the non-surgical therapy, it is required that the measured value of galectin still exceeded the threshold value after the surgical therapy (T₀)). When such a requirement is satisfied, a measured value (Cₙ) of galectin in a blood sample further collected thereafter (Tₙ) is compared with the measured value (Cₙ₋₁) and the threshold value (Cₜₕ). One example of this embodiment is schematically shown in FIG. 3.

(9) The method according to (5), comprising, prior to the step Pₙ (n≧2), the step Pₙ₋₁ of measuring a concentration of galectin-4 in a collected blood sample Sₙ₋₁ derived from the same individual and collected before collection of the blood sample Sₙ to acquire a measured value Cₙ₋₁[G4], and the step P₀ of measuring a concentration of galectin-4 in a collected blood sample S₀ derived from the same individual and collected before collection of the blood sample Sₙ₋₁ to acquire a measured value C₀[G4], wherein

the individual has undergone at least non-surgical therapy for colorectal cancer between the step P₀ and the step Pₙ₋₁, and has subsequently undergone the non-surgical therapy also between the step Pₙ₋₁ and the step Pₙ, and wherein

the measured value C₀[G4] acquired in the step P₀ exceeds the threshold value Cₜₕ[G4] of galectin-4, and the reference value C_{ref}[G4] compared with the measured value Cₙ[G4] in the step Pₙ is the threshold value Cₜₕ[G4] and the measured value Cₙ₋₁[G4].

The following is directed to a method using galectin-3 or galectin-4 as a “tumor screening marker”. This method comprises a comparison between a measured value of galectin in a collected blood sample and a threshold value of galectin.

(10) The method according to (4), wherein the reference value C_{ref}[G3/G4] of the colorectal cancer marker is a threshold value Cₜₕ[G3/G4] of the colorectal cancer marker.

The following is directed to a colorectal cancer marker galectin-1.

(11) Galectin-1 used as a tumor screening marker or a tumor progression marker for colorectal cancer.

The following is directed to a method of analyzing a concentration of galectin-1 in a collected blood sample. The analysis method according to the present invention comprises a comparison between a measured value of galectin in a collected blood sample and a reference value of galectin.

(12) A method of analyzing a galectin concentration in a collected blood sample, the method comprising the step Pₙ of measuring a concentration of galectin-1 in a collected blood sample Sₙ derived from an individual to acquire a measured value Cₙ[G1] and comparing the measured value Cₙ[G1] with a reference value C_{ref}[G1] of galectin-1, thereby analyzing the galectin concentration.

The following is directed to a method using galectin-1 as a “tumor screening marker”. This method comprises a comparison between a measured value of galectin in a collected blood sample and a threshold value of galectin.

(13) The method of analyzing a galectin concentration according to (12), wherein the reference value C_{ref}[G1] of galectin-1 is a threshold value Cₜₕ[G1] of galectin-1.

The following is directed to one embodiment of a method using galectin-1 as a “tumor progression marker”. This embodiment comprises a comparison between a measured value of galectin in a collected blood sample and a measured value of galectin in a blood sample previously collected and/or a threshold value of galectin.

(14) The method according to (12), further comprising, prior to the step Pₙ (n≧1), the step Pₙ₋₁ of measuring a concentration of galectin-1 in a collected blood sample Sₙ₋₁ derived from the same individual and collected before collection of the blood sample Sₙ to acquire a measured value Cₙ₋₁[G1], wherein

the reference value C_{ref}[G1] compared with the measured value Cₙ[G1] in the step Pₙ is selected from the group consisting of the measured value Cₙ₋₁[G1] and a threshold value Cₜₕ[G1] of galectin-1.

In the above (14), the individual may be one that has undergone treatment for colorectal cancer before the step Pₙ.

One example of the embodiment according to the above (14) is schematically shown in FIG. 1.

The following is directed to an embodiment of the method using galectin-1 as a “tumor progression marker”, in which the blood sample is derived from an individual that has been treated by at least surgery. This embodiment is applied to monitor an individual that has been confirmed to have no residual primary lesion of colorectal cancer after surgery (i.e., curability is A or B), and requires that a measured value of galectin in a blood sample collected before treatment for colorectal cancer exceeded a threshold value and a measured value of galectin in a blood sample collected after the treatment was below the threshold value. When such a requirement is satisfied, a measured value of galectin in a blood sample further collected thereafter is compared with the threshold value. One example of this embodiment is schematically shown in FIG. 2.

(15) The method according to (14), comprising, prior to the step Pₙ (n≧2), the step P₁ of measuring a concentration of galectin-1 in a collected blood sample S₁ derived from the same individual and collected before collection of the blood sample Sₙ to acquire a measured value C₁[G1], and the step P₀ of measuring a concentration of galectin-1 in a collected blood sample S₀ derived from the same individual and collected before collection of the blood sample S₁ to acquire a measured value C₀[G1], wherein

the individual has undergone treatment for colorectal cancer with surgery between the step P₀ and the step P₁,

the measured value C₀[G1] acquired in the step P₀ exceeds the threshold value Cₜₕ[G1] of galectin-1, and the measured value C₁[G1] acquired in the step P₁ is below the threshold value Cₜₕ[G1] and

the reference value C_{ref}[G1] compared with the measured value Cₙ[G1] in the step Pₙ is the threshold value Cₜₕ[G1].

(16) The method according to (15), wherein the individual has further undergone non-surgical therapy (e.g., radiation therapy or chemotherapy) for colorectal cancer between the step P₁ and the step Pₙ.

The following are directed to embodiments of the method using galectin-1 as a “tumor progression marker”, in which the blood sample is derived from an individual that has been treated by at least non-surgical therapy (e.g., radiation therapy or chemotherapy). In the following, the phrase “has undergone at least non-surgical therapy for colorectal cancer” includes both cases where the individual has undergone only non-surgical therapy, and where the individual has undergone surgical therapy before non-surgical therapy.

Further, the following embodiment requires that the non-surgical therapy is performed once and that a measured value (Cₙ₋₁) of galectin in a blood sample collected before treatment for colorectal cancer with the non-surgical therapy (Tₙ₋₁) exceeded a threshold value (in a case where surgical therapy has been performed before the non-surgical therapy, it is required that the measured value (Cₙ₋₁) of galectin still exceeded the threshold value after the surgical therapy (Tₙ₋₁)). When such a requirement is satisfied, a measured value (Cₙ) of galectin in a blood sample further collected thereafter (Tₙ) is compared with the measured value (Cₙ₋₁) and the threshold value (Cₜₕ).

(17) The method according to (14), wherein the individual has undergone at least non-surgical therapy for colorectal cancer between the step Pₙ₋₁ and the step Pₙ,

the measured value Cₙ₋₁[G1] acquired in the step Pₙ₋₁ exceeds the threshold value Cₜₕ[G1] of galectin-1, and the reference value C_{ref}[G1] compared with the measured value Cₙ[G1] in the step Pₙ is the threshold value Cₜₕ[G1] and the measured value Cₙ₋₁[G1].

On the other hand, the following embodiment requires that the non-surgical therapy is performed two or more times, and that a measured value of galectin in a blood sample collected before treatment for colorectal cancer with the non-surgical therapy (T₀) exceeded a threshold value (in a case where surgical therapy has been performed before the non-surgical therapy, it is required that the measured value of galectin still exceeded the threshold value after the surgical therapy (T₀)). When such a requirement is satisfied, a measured value (Cₙ) of galectin in a blood sample further collected thereafter (Tₙ) is compared with the measured value (Cₙ₋₁) and the threshold value (Cₜₕ). One example of this embodiment is schematically shown in FIG. 3.

(18) The method according to (14), comprising, prior to the step Pₙ (n≧2), the step Pₙ₋₁ of measuring a concentration of galectin-1 in a collected blood sample Sₙ₋₁ derived from the same individual and collected before collection of the blood sample Sₙ to acquire a measured value Cₙ₋₁[G1], and the step P₀ of measuring a concentration of galectin-1 in a collected blood sample S₀ derived from the same individual and collected before collection of the blood sample Sₙ₋₁ to acquire a measured value C₀[G1], wherein

the individual has undergone at least non-surgical therapy for colorectal cancer between the step P₀ and the step Pₙ₋₁, and has subsequently undergone the non-surgical therapy also between the step Pₙ₋₁ and the step Pₙ,

the measured value C₀[G1] acquired in the step P₀ exceeds a threshold value Cₜₕ[G1] of galectin-1, and the reference value C_{ref}[G1] compared with the measured value Cₙ[G1] in the step Pₙ is the threshold value Cₜₕ[G1] and the measured value Cₙ₋₁[G1].

As the threshold value used in the above method, a concentration value of galectin-3 and/or a concentration value of galectin-4 that indicate (s) high diagnostic accuracy is/are selected. Preferably, a galectin concentration value that indicates the following specificity is selected.

(19) The method according to any one of (5) to (10), wherein as the threshold value, a concentration value of galectin-3 and/or a concentration value of galectin-4 that indicate(s) a specificity of 80% or higher is/are selected.

The following is directed to an embodiment in which the colorectal cancer marker according to the present invention is used in combination with another tumor progression marker for colorectal cancer.

(20) The method according to any one of (5) to (9), wherein the step Pₙ further comprises analysis performed by measuring a concentration of another tumor progression marker for colorectal cancer in the collected blood sample Sₙ to acquire a measured value Cₙ[other] and comparing the measured value Cₙ[other] with a reference value C_{ref}[other] of the another tumor progression marker for colorectal cancer.

(21) The method according to (20), wherein the another tumor progression marker for colorectal cancer is selected from the group consisting of carcinoembryonic antigen (CEA) and CA19-9.

The following is directed to an embodiment in which galectin-3 and/or galectin-4 are/is measured by a specific method.

(22) The method according to any one of (4) to (10), wherein the measurement is performed by an immunoassay using a detection antibody selected from the group consisting of galectin-3 antibody and galectin-4 antibody that are labeled with a fluorescent compound and/or an enzyme protein.

In the above method, the enzyme protein may be selected from the group consisting of peroxidase, alkaline phosphatase, and β-galactosidase.

As the threshold value used in the above method, a concentration value of galectin-1 that indicates high diagnostic accuracy is selected. Preferably, a galectin concentration value that indicates the following specificity is selected.

(23) The method according to any one of (13) to (18), wherein as the threshold value, a concentration value of galectin-1 that indicates a specificity of 80% or higher is selected.

The following is directed to an embodiment in which the colorectal cancer marker according to the present invention is used in combination with another tumor progression marker for colorectal cancer.

(24) The method according to any one of (14) to (18), wherein the step Pₙ further comprises analysis performed by measuring a concentration of another tumor progression marker for colorectal cancer in the collected blood sample Sₙ to acquire a measured value Cₙ[other] and comparing the measured value Cₙ[other] with a reference value C_{ref}[other] of the another tumor progression marker for colorectal cancer.

(25) The method according to (24), wherein the another tumor progression marker for colorectal cancer is selected from the group consisting of carcinoembryonic antigen (CEA) and CA19-9.

The following is directed to an embodiment in which galectin-1 is measured by a specific method.

(26) The method according to any one of (12) to (21), wherein the measurement is performed by an immunoassay using, as a detection antibody, galectin-1 antibody labeled with a fluorescent compound and/or an enzyme protein.

In the above method, the enzyme protein may be selected from the group consisting of peroxidase, alkaline phosphatase, and β-galactosidase.

The following is directed to a kit for detecting the colorectal cancer marker according to the present invention.

(27) A colorectal cancer marker detection kit comprising a detection antibody selected from the group consisting of galectin-1 antibody, galectin-3 antibody, and galectin-4 antibody that are labeled with a fluorescent compound and/or an enzyme protein.

(28) The kit according to the above (27), wherein the enzyme protein is selected from the group consisting of peroxidase, alkaline phosphatase, and β-galactosidase.

### Effects of the Invention

According to the present invention, it is possible to provide a tumor screening marker that can be actually used in clinical practice to detect colorectal cancer, a tumor progression marker that can complement CEA or CA19-9, and a prognostic prediction marker. More specifically, galectin-1, galectin-3, and galectin-4 can be provided as tumor screening markers, galectin-1 and galectin-4 can be provided as tumor progression markers, and galectin-4 can be provided as a prognostic prediction marker.

Further, according to the present invention, it is possible to provide a method of analyzing a collected blood sample using such a marker.

Particularly, the use of galectin-1 as a marker for cancer detection makes it possible to achieve a high positive rate among patients with early-stage cancer. Further, the combined use of galectin-1 or galectin-4 with an existing colorectal cancer marker makes it possible to improve a patient capture rate (i.e., a positive rate) as compared to when only the existing colorectal cancer marker is used for cancer detection.

### BRIEF DESCRIPTION OF THE DRAWINGS

FIG. 1 is a diagram schematically showing an embodiment using a tumor progression marker according to the present invention.

FIG. 2 is a diagram schematically showing an embodiment in which the tumor progression marker according to the present invention is used for a patient who has been treated by surgery.

FIG. 3 is a diagram schematically showing an embodiment in which the tumor progression marker according to the present invention is used for a patient under treatment with non-surgical therapy other than surgery (e.g., with radiation therapy or chemotherapy).

FIG. 4 shows the results of comparison of the concentration of galectin in collected blood samples between a healthy individual group and a colorectal cancer patient group. FIG. 4(A) shows the results of galectin-1, FIG. 4(B) shows the results of galectin-3, and FIG. 4(C) shows the results of galectin-4. A box in each box plot represents the range from 25th to 75th percentile of concentration distribution of all the samples, horizontal lines represent the range from 10th to 90th percentile of concentration distribution of all the samples, and a horizontal line in the box represents a median concentration in each group (colorectal cancer patient group (CRC) or healthy individual group (Control)).

FIG. 5 shows the results of comparison of the concentration of galectin in collected blood samples among a healthy individual group and colorectal cancer patient groups at different stages of cancer. FIG. 5(A) shows the results of galectin-1, FIG. 5(B) shows the results of galectin-3, and FIG. 5(C) shows the results of galectin-4. Each box plot represents the range from 10th to 90th percentile of concentration distribution of all the samples, and a horizontal line in the box represents a median concentration in each group (colorectal cancer patient group (CRC) or healthy individual group (Control)).

FIG. 6 shows ROC curves showing the discrimination between colorectal cancer patients and healthy individuals based on the concentration of galectin in collected blood samples. The vertical axis represents a positive rate and the horizontal axis represents a false-positive rate (100-specificity). FIG. 6(A) shows a ROC curve for galectin-1, FIG. 6(B) shows a ROC curve for galectin-3, and FIG. 6(C) shows a ROC curve for galectin-4.

FIG. 7 shows the results of comparison of the concentration of galectin in blood samples collected before and after surgery from individuals whose galectin concentration before surgery exceeded a threshold value (i.e., who were positive for galectin). FIG. 7(A) shows the results of galectin-1, FIG. 7(B) shows the results of galectin-3, and FIG. 7(C) shows the results of galectin-4. Plots connected by a line represent the concentrations of galectin in blood samples collected from the same individual before and after surgery.

FIG. 8 shows the results of comparison of patient capture rates (i.e., positive rates) of cancer patient groups in different disease states between when galectin-1 or galectin 4 was used as a marker and when only CEA or CA19-9 was used as a marker. FIG. 8(A) shows the results of comparison between CEA and galectin-4, FIG. 8(B) shows the results of comparison between CEA and galectin-1, FIG. 8 (C) shows the results of comparison between CA19-9 and galectin-4, and FIG. 8(D) shows the results of comparison between CA19-9 and galectin-1. And, FIG. 8(A) shows also the positive rates when CEA and galectin-4 were used in combination and FIG. 8(C) shows also the positive rates when CA19-9 and galectin-4 were used in combination (in both cases, a case where at least one of the marker levels exceeded a threshold value was regarded as a positive case).

### MODES FOR CARRYING OUT THE INVENTION

### 1. Colorectal Cancer Marker

The present invention provides galectin-1, galectin-3, and galectin-4 as colorectal cancer markers. Each of these markers shows a difference in concentration thereof in a collected blood sample between a colorectal cancer patient group and a healthy individual group, or among colorectal cancer patient groups different in the disease state (size) of colorectal cancer. That is, these markers show an increase in expression in colorectal cancer.

The colorectal cancer markers provided by the present invention can be used as a tumor progression marker, a tumor screening marker, and a prognostic prediction marker.

More specifically, galectin-1 and galectin-4 can be used as tumor progression markers. Galectin-1, galectin-3, and galectin-4 can be used as tumor screening markers. Galectin-4 can be used as a prognostic prediction marker.

### 2. Collected Blood Sample

The colorectal cancer marker according to the present invention can be detected/analyzed in a collected blood sample. Therefore, the concentration of the colorectal cancer marker in a collected blood sample is analyzed by a method according to the present invention.

A collected blood sample is a sample directly subjected to galectin concentration measurement, and includes whole blood, blood plasma, blood serum, and the like. The blood sample can be prepared by appropriately treating whole blood collected from an individual. Treatment performed to prepare a collected blood sample from collected whole blood is not particularly limited as long as it is clinically acceptable. For example, centrifugal separation may be performed. The collected blood sample subjected to galectin concentration measurement may be one that has been suitably stored at low temperatures such as frozen in the course of or after its preparation step. It is to be noted that in the present invention, the collected blood sample is discarded without being returned to an individual as it source.

Examples of the individual as a source of the collected blood sample include those who require the diagnosis of presence of colorectal cancer, colorectal cancer patients who require a disease state diagnosis during follow-up after treatment, and those who require a prognostic prediction.

### 3. Analysis of Concentration of Colorectal Cancer Marker in Collected Blood Sample

According to the present invention, the concentration of the cancer marker in a blood sample is analyzed by a comparison between a measured value and a reference value of the cancer marker. In order to more accurately perform the analysis, the comparison between the measured value and the reference value is preferably performed based on collected blood samples prepared under the same conditions (e.g., pretreatment conditions, storage conditions).

The method according to the present invention comprises the step Pₙ of measuring the concentration of the colorectal cancer marker in a collected blood sample Sₙ derived from blood collected at some point in time to acquire a measured value Cₙ of the colorectal cancer marker and comparing the measured value Cₙ of the colorectal cancer marker with a reference value C_{ref} of the colorectal cancer marker.

### 4. Reference Value

The reference value C_{ref} is a value used as a criterion for determining the disease state or the like of colorectal cancer. As described above, the colorectal cancer marker according to the present invention shows a difference in concentration thereof in a collected blood sample between a colorectal cancer patient group and a healthy individual group, or among colorectal cancer patient groups different in the disease state (size) of colorectal cancer. Therefore, the setting of an appropriate reference value C_{ref} makes it possible to effectively discriminate between these groups.

When the measured value Cₙ is higher than the reference value C_{ref}, it is possible to judge that there is a high possibility that the disease state is severe, and on the other hand, when the measured value Cₙ is lower than the reference value C_{ref}, it is possible to judge that there is a high possibility that the disease state is not severe.

### [4-1. Threshold Value]

One specific example of the reference value is a threshold value Cₜₕ specific to each of the colorectal cancer markers. The threshold value Cₜₕ used in the present invention can be previously set for each kind of galectin depending on race, age, etc. The threshold value Cₜₕ can be set by reference to respective measured values of a healthy individual group and a colorectal cancer patient group acquired by measuring the amounts of the colorectal cancer marker present in respective collected blood samples derived from individuals belonging to the healthy individual group and individuals belonging to the colorectal cancer patient group by a measurement method that will be described later.

Alternatively, the threshold value Cₜₕ may be set by reference to respective measured values of patient groups in different disease states of colorectal cancer acquired by measuring the amounts of the colorectal cancer marker present in respective collected blood samples derived from colorectal cancer patients by a measurement method that will be described later. It is to be noted that the difference in the disease state of colorectal cancer can be represented by, for example, the difference in the stage of cancer progression determined by the total amount of cancer present in the body or the extent of metastasis. The stage of cancer progression can be determined based on, for example, TMN classification. More specifically, primary cancer is referred to as Stage 0 (cancer in situ), Stage I, and Stage II; lymph node metastatic cancer is referred to as Stage III; and distal metastatic cancer is referred to as Stage IV. In this specification, the colorectal cancers from Stage 0 to Stage IV are collectively called colorectal cancer in the absence of a description of the stage of cancer.

As the threshold value Cₜₕ, a cut-off value that yields high diagnostic accuracy is selected. Preferably, the threshold value Cₜₕ can be appropriately selected by those skilled in the art from cut-off values that yield a specificity of 80% or higher. The upper limit of the specificity is not particularly limited, but may be, for example, 95%.

A method for setting the threshold value Cₜₕ is appropriately selected by those skilled in the art. One example of the method is ROC Curve (Receiver Operating Characteristic Curve) analysis.

### [4-2. Previous Measured Value]

Another specific example of the reference value may be a measured value of the colorectal cancer marker in a blood sample previously collected from the same individual.

### 5. Use of Colorectal Cancer Marker for Purposes

A determination as to which of the threshold value and the previous measured value is used as the reference value is made depending on the kind of colorectal cancer marker used and the intended use of the colorectal cancer marker.

### [5-1. Use of Tumor Screening Marker]

When the tumor screening marker (i.e., galectin-1, galectin-3, or galectin-4) is used, a reference value C_{ref} of the tumor screening marker is used as a criterion for discrimination between collected blood samples derived from colorectal cancer patients and collected blood samples derived from healthy individuals. More specifically, the reference value C_{ref} of the tumor screening marker is a threshold value Cₜₕ of the tumor screening marker.

Therefore, when a measured value Cₙ of the tumor screening marker is higher than the reference value C_{ref}, it is possible to judge that there is a high possibility that an individual as a source of the collected blood sample Sₙ has colorectal cancer (i.e., the individual is highly suspected of having colorectal cancer). On the other hand, when a measured value Cₙ of the tumor screening marker is lower than the reference value C_{ref}, it is possible to judge that there is a high possibility that an individual as a source of the collected blood sample Sₙ is healthy (i.e., the individual has a low probability of colorectal cancer)

### [5-2. Use of Prognostic Prediction Marker]

When the prognostic prediction marker (i.e, galectin-4) is used, a reference value of prognostic prediction marker is used as a criterion for discrimination between collected blood samples derived from colorectal cancer patients whose prognosis is poor and collected blood samples derived from colorectal cancer patients whose prognosis is not poor. More specifically, the reference value C_{ref}[G4] of the prognostic prediction marker is a threshold value Cₜₕ[G4] of the prognostic prediction marker.

Therefore, when a measured value C₀[G4] of the prognostic prediction marker is higher than the reference value C_{ref}[G4] (i.e., than the threshold value Cₜₕ[G4]) it is possible to judge that there is a high possibility that an individual as a source of the collected blood sample S₁ has a poor prognosis. On the other hand, when a measured value C₀[G4] of the prognostic prediction marker is lower than the reference value C_{ref}[G4] (i.e., than the threshold value Cₜₕ[G4]), it is possible to judge that there is a low possibility that an individual as a source of the collected blood sample S₀ has a poor prognosis.

### [5-3. Use of Tumor Progression Marker]

When the tumor progression marker (i.e., galectin-4 or galectin-1) is used, a reference value of the tumor progression marker is used as a criterion for evaluation of collected blood samples that are derived from the same individual but are collected at different times during the course of a disease (more specifically, at different stages of colorectal cancer progression and the amount of cancer present in the body). Therefore, when the tumor progression marker is used, the marker level of a collected blood sample derived from the same individual as a collected blood sample Sₙ subjected to the step Pₙ and collected before the collection of the blood sample Sₙ is measured.

Here, measured values (concentrations) of the colorectal cancer marker in collected blood samples (S₀, S₁, S₂, S₃, . . . , Sₙ₋₁, Sₙ) derived from blood collected from a colorectal cancer patient serially from some point Tₙ in time (T₀, T₁, T₂, T₃, . . . , Tₙ₋₁, Tₙ) are defined as C₀, C₁, C₂, C₃, . . . , Cₙ₋₁, Cₙ, respectively.

The method using the tumor progression marker is applied when it has already been judged that there is a high possibility that an individual as a source of a collected blood sample has colorectal cancer (the individual is suspected of having colorectal cancer). Such a judgment can be made using the above-described tumor screening marker according to the present invention. For example, galectin-1 or galectin-4 can be used. A collected blood sample derived from an individual whose measured value of the tumor screening marker was judged to be higher than the threshold value of the tumor screening marker (which is collected after the collection of a blood sample subjected to the judgment using the tumor screening marker) may be subjected to analysis using the tumor progression marker.

Further, the method using the tumor progression marker according to the present invention is preferably applied when an individual whose measured value of the tumor screening marker in a blood sample was judged to be higher than the threshold value of the tumor screening marker has undergone treatment for colorectal cancer between the collection of the blood sample subjected to the judgment and the collection of a blood sample to be subjected to analysis using the tumor progression marker.

Examples of the treatment for colorectal cancer include surgery and non-surgical therapy. Examples of the non-surgical therapy include non-invasive therapies such as chemotherapy and radiation therapy. Such non-surgical therapy may be performed only once, but may be often performed two or more times continuously (continuous therapy). When such treatment is performed, evaluation and follow-up of therapeutic effects can be performed by the method using the tumor progression marker according to the present invention.

### [5-3-1. Embodiment Using Tumor Progression Marker]

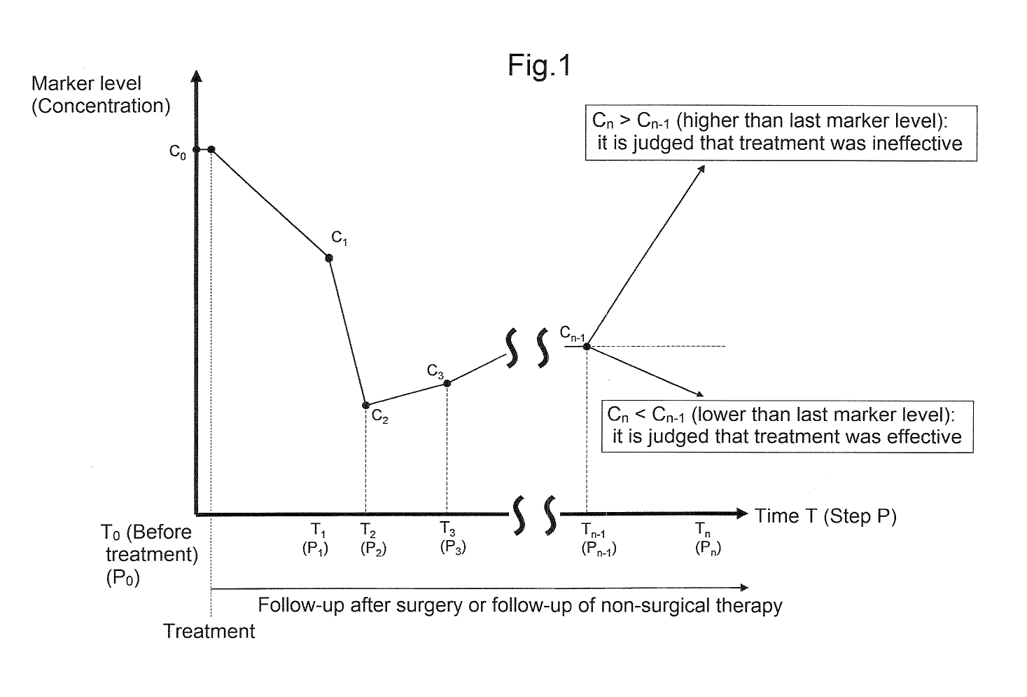

One example of an embodiment using the tumor progression marker is schematically shown in FIG. 1.

Prior to a step Pₙ (n≧1), a step Pₙ₋₁ is performed to measure the concentration of the tumor progression marker in a collected blood sample Sₙ₋₁ derived from the same individual as a collected blood sample Sₙ and collected at a time Tₙ₋₁ before a time Tₙ when the blood sample Sₙ is collected to acquire a measured value Cₙ₋₁. The measured value Cₙ₋₁ is used as a reference value C_{ref} in the step Pₙ performed thereafter. That is, in the step Pₙ, the concentration of the tumor progression marker in the blood sample Sₙ derived from the same individual as the blood sample Sₙ₋₁ and collected after the collection of the blood sample Sₙ₋₁ is measured to acquire a measured value Cₙ, and the measured value Cₙ is compared with the measured value Cₙ₋₁ as a reference value C_{ref}.

Therefore, when the measured value Cₙ is higher than the reference value C_{ref} (i.e., than the measured value Cₙ₋₁), it is possible to judge that there is a high possibility that the disease state of the individual as a source of the collected blood sample Sₙ is worse at the time Tₙ than at the time Tₙ₋₁. On the other hand, when the measured value Cₙ is lower than the reference value C_{ref} (i.e., than the measured value Cₙ₋₁), it is possible to judge that there is a high possibility that the disease state of the individual as a source of the collected blood sample Sₙ is better at the time Tₙ than at the time Tₙ₋₁.

When treatment for colorectal cancer has been performed before the time Tₙ, the effects of the treatment can be evaluated in the following manner. For example, in a case where non-surgical therapy for colorectal cancer has been performed between the time Tₙ₋₁ and the time Tₙ, when the measured value Cₙ is higher than the reference value C_{ref} (i.e., than the measured value Cₙ₋₁), it is possible to judge that there is a high possibility that the treatment was not effective for the individual as a source of the collected blood sample Sₙ at the time Tₙ, and when the measured value Cₙ is lower than the reference value C_{ref} (i.e., than the measured value Cₙ₋₁), it is possible to judge that there is a high possibility that the treatment was effective for the individual as a source of the collected blood sample Sₙ at the time Tₙ.

In this way, it is possible to follow-up the effects of non-surgical therapy such as radiation therapy or chemotherapy.

### [5-3-2. Specific Embodiment 1 Using Tumor Progression Marker]

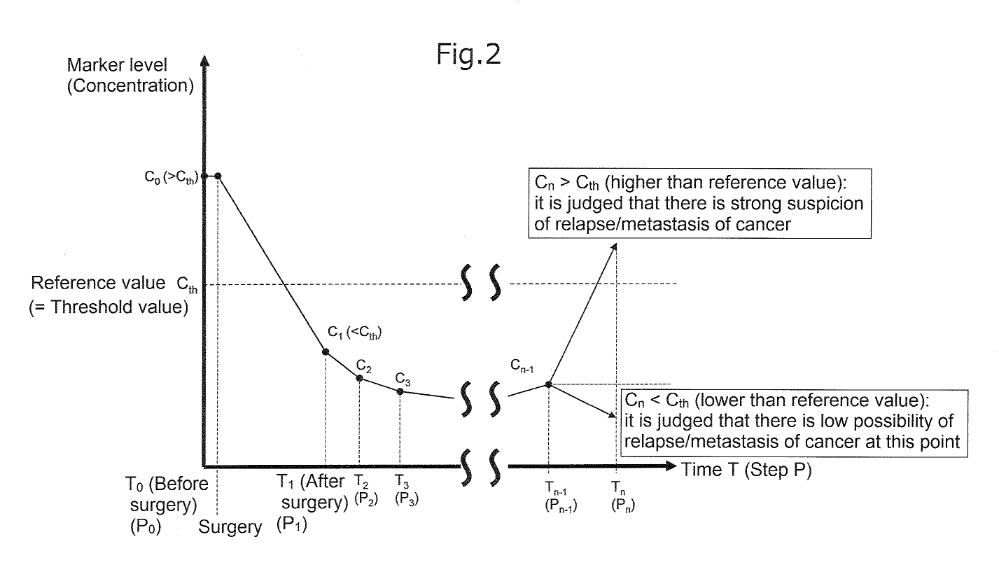

One example of a specific embodiment using the tumor progression marker, which is applied to a case where surgery has been used as treatment, is schematically shown in FIG. 2.

This embodiment is applied to a case where surgery has been performed as treatment for colorectal cancer between a time T₀ and a time T₁ based on the premise that it has been confirmed that there is no residual primary lesion of colorectal cancer after surgery (that is, curability is A or B). Further, this embodiment is performed when it has been confirmed that a measured value C₀ of the tumor progression marker in a blood sample S₀ collected at the time T₀ before surgery exceeded a threshold value Cₜₕ of the tumor progression marker, and a measured value C₁ of the tumor progression marker in a blood sample S₁ collected at the time T₁ after surgery was below the threshold value Cₜₕ of the tumor progression marker (i.e, the amount of colorectal cancer present in the body has been reduced or colorectal cancer has disappeared).

As described above, in the step P₁ performed after treatment, the concentration of the tumor progression marker in the collected blood sample S₁ is measured and the measured value C₁ below the threshold value Cₜₕ of the tumor progression marker is acquired. Then, in a step Pₙ performed thereafter, the concentration of the tumor progression marker in a blood sample Sₙ collected from the same individual as the blood sample S₁ at a time Tₙ after the time T₁ is measured to acquire a measured value Cₙ, and then the measured value Cₙ is compared with the threshold value Cₜₕ as a reference value C_{ref}.

When the measured value Cₙ higher than the reference value C_{ref} (i.e., than the threshold value Cₜₕ), it is possible to judge that the individual as a source of the blood sample Sₙ is suspected of relapse or metastasis of colorectal cancer at the time Tₙ. On the other hand, when the measured value Cₙ is lower than the reference value C_{ref} (i.e., than the threshold value Cₜₕ), it is possible to judge that there is a low possibility that the individual as a source of the blood sample Sₙ has a relapse or metastasis of colorectal cancer at the time Tₙ.

Therefore, it is possible to perform follow-up to detect relapse and metastasis of colorectal cancer after surgery. It is to be noted that after treatment with surgery, only follow-up may be performed without any particular treatment or non-surgical therapy may be performed. The follow-up for detection of relapse and metastasis of colorectal cancer performed after treatment with surgery may be the above-described follow-up performed without any particular treatment or may be performed by non-surgical therapy.

### [5-3-3. Specific Embodiment 2 Using Tumor Progression Marker]

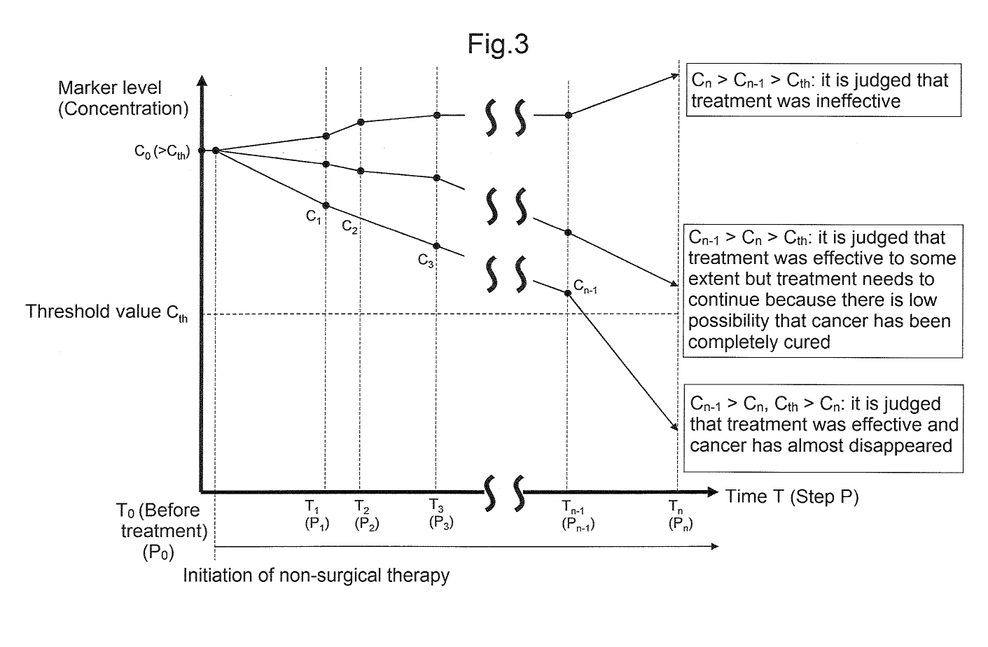

One example of a specific embodiment using the tumor progression marker, which is applied to a case where non-surgical therapy has been used as treatment, is schematically shown in FIG. 3.

This embodiment is intended to be applied to a case where at least initial non-surgical therapy for colorectal cancer has been performed between a step P₀ and a step Pₙ₋₁ and non-surgical therapy has been subsequently performed also between the step Pₙ₋₁ and a step Pₙ. Further, this embodiment is based on the premise that it has been confirmed that a measured value C₀ of the tumor progression marker in a blood sample S₀ collected at a time T₀ before the initial treatment with non-surgical therapy exceeded a threshold value Cₜₕ of the tumor progression marker. When surgical therapy has been performed before the initial non-surgical therapy, this embodiment is applied to a case where the measured value Cₙ₋₁ of the tumor progression marker still exceeded the threshold value Cₜₕ after the surgical therapy (T₀).

More specifically, as in the case of the above-described embodiment 1 using the tumor progression marker, a step Pₙ₋₁ is performed to measure the concentration of the tumor progression marker in a collected blood sample Sₙ₋₁ derived from the same individual as a collected blood sample Sₙ and collected at a time Tₙ₋₁ before a time Tₙ when the blood sample Sₙ is collected to acquire a measured value Cₙ₋₁. This measured value Cₙ₋₁ can be used as a reference value C_{ref} in a step Pₙ performed thereafter.

In a step Pₙ, a measured value Cₙ is compared with both the measured value Cₙ₋₁ and the threshold value Cₜₕ as a reference value C_{ref}.

For example, a comparison between the measured value Cₙ and the reference value Cₙ₋₁ makes it possible to determine whether the treatment was effective or not. More specifically, when the measured value Cₙ is higher than the reference value Cₙ₋₁, it is possible to judge that there is a high possibility that the treatment was not effective for the individual as a source of the collected blood sample Sₙ at the time Tₙ. On the other hand, when the measured value Cₙ is lower than the reference value Cₙ₋₁, it is possible to judge that there is a high possibility that the treatment was effective for the individual as a source of the blood sample Sₙ at the time Tₙ.

Further, a comparison between the measured value Cₙ and the threshold value Cₜₕ makes it possible to determine whether cancer is present or not. More specifically, when the measured value Cₙ is higher than the threshold value Cₜₕ, there is a high possibility that the individual as a source of the collected blood sample Sₙ still has cancer (i.e., cancer has not disappeared) at the time Tₙ. On the other hand, when the measured value Cₙ is lower than the threshold value Cₜₕ, there is a high possibility that the individual as a source of the blood sample Sₙ no longer has cancer (i.e., cancer has disappeared) at the time Tₙ.

Therefore, a combination of both the comparisons makes it possible to determine whether treatment needs to continue or not. For example, when the measured value Cₙ is higher than both the reference value Cₙ₋₁ and the threshold value Cₜₕ, it is possible to judge that the treatment was ineffective. On the other hand, when the measured value Cₙ is lower than the reference value Cₙ₋₁ but higher than the threshold value Cₜₕ, it is possible to judge that the treatment was effective but cancer has not been completely cured and therefore the treatment needs to continue. Moreover, when the measured value Cₙ is lower than both the reference value Cₙ₋₁ and the threshold value Cₜₕ, it is possible to judge that cancer has almost disappeared due to therapeutic effects.

As described above, a comparison between the measured value Cₙ and the measured value Cₙ₋₁ makes it possible to follow-up the effects of treatment for cancer. Further, a comparison between the measured value Cₙ and the threshold value Cₜₕ also makes it possible to make a determination as to whether treatment needs to continue or not.

It is to be noted that this embodiment has been described above with reference to a case shown in FIG. 3 where non-surgical therapy is continuously performed two or more times, but can be applied also to a case where non-surgical therapy is performed only once.

When non-surgical therapy is performed only once, this embodiment is intended to be applied to a case where non-surgical therapy for colorectal cancer has been performed only once between the step Pₙ₋₁ and the step Pₙ. Further, this embodiment is based on the premise that it has been confirmed that a measured value Cₙ₋₁ of the tumor progression marker in a blood sample Sₙ₋₁ collected at a time Tₙ₋₁ before treatment with one-time non-surgical therapy exceeded a threshold value Cₜₕ of the tumor progression marker. When surgical therapy has been performed before the one-time non-surgical therapy, this embodiment is applied to a case where the measured value Cₙ₋₁ of the tumor progression marker still exceeded the threshold value Cₜₕ after the surgical therapy (Tₙ₋₁). Those skilled in the art can implement the present invention also when non-surgical therapy is performed only once by reference to the above-described case where non-surgical therapy is continuously performed two or more times.

[5-4. Combined Use with Another Colorectal Cancer Marker]

The method according to the present invention using the tumor progression marker is useful also when the tumor progression marker in the present invention is used to complement another tumor progression marker for colorectal cancer. Examples of the another tumor progression marker for colorectal cancer include carcinoembryonic antigen (CEA), CA19-9, and the like.

In this case, the step Pₙ further includes measuring the concentration of another tumor progression marker for colorectal cancer in the collected blood sample Sₙ to acquire a measured value Cₙ[other], and comparing the measured value Cₙ[other] with a reference value C_{ref}[other] of the another tumor progression marker.

As a result, there is a case where, even when the collected blood sample is derived from a colorectal cancer patient, it is judged that the measured value Cₙ[other] is below the reference value C_{ref}[other] (i.e., the patient is not suspected of having colorectal cancer). However, even in such a case, there is a case where the blood sample is diagnosed as positive for the tumor progression marker according to the present invention. In this case, the collected blood sample false-negative for the another tumor progression marker can be correctly diagnosed by the tumor progression marker according to the present invention.

On the other hand, when the blood sample is diagnosed as negative also for the tumor progression marker according to the present invention, this diagnosis can support that the negative diagnostic result obtained by the another tumor progression marker (i.e., the patient is not suspected of having colorectal cancer) is true.

In this way, the tumor progression marker according to the present invention can complement another tumor progression marker for colorectal cancer.

### [5-5. Measurement Method]

The colorectal cancer marker according to the present invention is preferably measured by a test based on biospecific affinity. The test based on biospecific affinity is a method well known to those skilled in the art and is not particularly limited, but is preferably an immunoassay. Specific examples of the immunoassay include competitive and non-competitive assays such as western blotting, radioimmunoassay, ELISA (Enzyme-Linked ImmunoSorbent Assay: sandwich immunoassay, competitive assay, and direct binding assay are all included), immunoprecipitation, precipitation reaction, immunodiffusion, immunoagglutination, complement-binding reaction analysis, immunoradiometric assay, fluorescence immunoassay, and protein A immunoassay. In the immunoassay, an antibody that binds to the colorectal cancer marker in a collected blood sample is detected.

The antibody that binds to the colorectal cancer marker is appropriately determined by those skilled in the art using the colorectal cancer marker. For example, a labeled galectin antibody (monoclonal antibody or polyclonal antibody) is used. A label in the labeled galectin antibody may be a fluorescent compound and/or an enzyme protein. As the fluorescent compound and the enzyme protein, those acceptable in a measurement system using an antibody are appropriately selected by those skilled in the art. For example, the enzyme protein may be selected from the group consisting of peroxidase, alkaline phosphatase, and β-galactosidase.

Preferably, an antibody against the colorectal cancer marker protein may be selected from the group consisting of galectin-1 antibody labeled with alkaline phosphatase, galectin-3 antibody labeled with peroxidase, and galectin-4 antibody labeled with peroxidase.

It is to be noted that a specific protocol for preparation and labeling of the galectin antibody can be easily selected by those skilled in the art.

The measurement of the colorectal cancer marker is performed by bringing an antibody blood sample into contact with an antibody under the condition that a colorectal cancer marker protein to be measured and an antibody against the colorectal cancer marker protein can form an immunocomplex.

A specific protocol for the immunoassay can be easily selected by those skilled in the art.

One example of the protocol is as follows. A capture antibody is, for example, adsorbed onto a substrate or a well inner wall to obtain a solid phase-capture antibody. As the capture antibody, a galectin polyclonal (or monoclonal) antibody that recognizes an epitope on a galectin protein different from that recognized by the above-described labeled galectin antibody is preferably used. The concentration of a capture antibody solution used to obtain a solid phase capture antibody is appropriately determined by those skilled in the art using the protocol. For example, the concentration of the capture antibody solution may be set to a value in the range of 1 to 10 μg/mL as recommended in the protocol of IMMUNO-TEK ELISA Construction System (ZeptoMetrix) used to construct an ELISA kit. One example of the concentration of the capture antibody solution may be 5 μg/mL.

A collected blood sample is added to the solid phase-capture antibody under the condition that the capture antibody and galectin in the blood sample can form an immunocomplex. If necessary, the blood sample may be appropriately diluted before subjected to the above treatment. The dilution factor at the time when galectin-1, galectin-3, or galectin-4 is detected is appropriately determined by those skilled in the art using the protocol. For example, the dilution factor can be determined in the range of 1- to 20-fold, preferably in the range of 5- to 10-fold in consideration of the kind of sample to be measured or other conditions. For example, when galectin-1 is detected, the dilution factor may be set to 10-fold, when galectin-3 is detected, the dilution factor may be set to 5-fold, and when galectin-4 is detected, the dilution factor may be set to 5-fold.

The substrate or the well is washed, and then the above-described labeled galectin antibody is added under the condition that galectin derived from the collected blood sample and bound to the capture antibody, and the labeled galectin antibody can form an immunocomplex. The concentration of the labeled galectin antibody added is appropriately determined by those skilled in the art using the protocol in consideration of the kind of sample to be measured or other conditions. For example, the concentration of the labeled galectin antibody can be determined in the range of 0.1 to 10 μg/mL, preferably in the range of 0.1 to 2 μg/mL. The concentration of the labeled galectin-1 antibody may be set to, for example, 0.5 μg/mL, the concentration of the labeled galectin-3 antibody may be set to, for example, 0.1 μg/mL, and the concentration of the labeled galectin-3 antibody may be set to, for example, 0.2 μg/mL.

Then, the substrate or the well is washed, and a signal derived from the labeled galectin antibody bound to galectin is detected. For example, when the antibody is labeled with a fluorescent compound, the amount of fluorescence derived from the label can be measured. Further, when the antibody is labeled with an enzyme protein, a signal can be measured by adding a substrate for the enzyme protein and detecting chemiluminescence derived from a compound obtained by decomposition of the substrate.

### 6. Kit for Detecting Colorectal Cancer Marker in Collected Blood Sample

The present invention provides a colorectal cancer marker detection kit comprising a detection antibody selected from the group consisting of labeled galectin-1 antibody, labeled galectin-3 antibody, and labeled galectin-4 antibody. The labeled galectin is a labeled galectin with a substance selected from the group consisting of a fluorescent compound, peroxidase, alkaline phosphatase, and β-galactosidase. The colorectal cancer marker detection kit according to the present invention can be used to perform the above-described colorectal cancer marker analysis.

The labeled galectin-1 antibody may be galectin-1 labeled with alkaline phosphatase, the labeled galectin-3 antibody may be galectin-3 antibody labeled with peroxidase, and the labeled galectin-4 antibody may be galectin-4 labeled with peroxidase.

Each of these detection antibodies may be provided as a solution prepared to have the above-described concentration.

The colorectal cancer marker detection kit may include, as an additional item, the above-described capture antibody selected from the group consisting of polyclonal (or monoclonal) anti-galectin-1, polyclonal (or monoclonal) anti-galectin-3, and polyclonal (or monoclonal) anti-galectin-4. The capture antibody may be provided as a solution prepared to have the above-described concentration or as a solid phase on the surface of a substrate or on the inner wall of a well.

### EXAMPLES

### Reference Example 1

### Preparation of Plasma Sample

In the following examples, plasma samples were prepared in the following manner. About 15 mL of blood per person was collected in a BD Vacutainer CPTTM tube. After blood collection, the collected blood was immediately centrifuged (1,700×g, 4° C., 20 min) to obtain a supernatant as a plasma component (about 5 mL). The obtained plasma sample was stored at −80° C.

The plasma sample was thawed before measurement and diluted at a dilution factor shown in Table 1 below to prepare a collected blood sample used to measure a galectin concentration.

### Example 1

### ELISA Measurement System

ELISA measurement systems for galectin detection (galectin-1 ELISA, galectin-3 ELISA, and galectin-4 ELISA) were prepared using a capture antibody, a labeled detection antibody, and a detection reagent shown in Table 1. The labeled detection antibody was obtained by labeling a non-labeled detection antibody with a labeling protein (alkaline phosphatase or peroxidase).

A solid phase antibody was prepared by adding a capture antibody solution (5 μg/mL, 100 μL) to each of the wells of a 96-well plate (Maxisorp). The solid phase capture antibody was obtained using IMMUNO-TEK ELISA Construction System (ZeptoMetrix, Buffalo, N.Y.)

TABLE 1

| | Galectin-1 ELISA | Galectin-3 ELISA | Galectin-4 ELISA |
|---|---|---|---|
| Recombinant protein | Abnova, Taiwan | R&D systems, Minneapolis, MN | R&D systems, Minneapolis, MN |
| manufacturer | | | |
| Capture antibody | Polyclonal anti-galectin-1 | Polyclonal anti-galectin-3 | Polyclonal anti-galectin-4 |
| (manufacturer) | antibody (R & D systems) | antibody (R & D systems) | antibody (R & D systems) |
| Concentration of | 5 μg/mL | 5 μg/mL | 5 μg/mL |
| Capture antibody | | | |
| Detection antibody | Monoclonal anti-galectin-1 | Monoclonal anti-galectin-3 | Polyclonal anti-galectin-4 |
| (manufacturer) | antibody (Abnova) | antibody (R & D systems) | antibody (R & D systems) |
| Concentration of | 0.5 μg/mL | 0.1 μg/mL | 0.2 μg/mL |
| Labeled detection | | | |
| antibody | | | |
| Labeled protein | Alkaline Phosphatase Labeling | Peroxidase Labeling | Peroxidase Labeling |
| (kit, manufacturer) | (Alkaline Phosphatase Labeling | (Peroxidase Labeling Kit-SH, | (Peroxidase Labeling Kit-SH, |
| | Kit-NH2, Dojindo Molecular | Dojindo Molecular | Dojindo Molecular Technologies, Inc.) |
| | Technologies, Inc.) | Technologies, Inc.) | |
| Dilution factor of | 10-fold | 10-fold | 20-fold |
| Measurement sample | | | |
| Detection reagent | AP-Blue SpectraFX Microwell | TMB Two Component HRP | TMB Two Component HRP Microwell |
| (manufacturer) | and/or | Microwell Substrate | Substrate |
| | Membrane Substrate | (BioFX Laboratories, Inc., Owings | (BioFX Laboratories, Inc., Owings Mills) |
| | (BioFX Laboratories, Inc., | Mills) | |
| | Owings Mills) | | |

### <Cross-Reactivity>

As standard samples for use in an antibody cross-reactivity test, recombinant proteins (Recombinant Human Galectins) shown in Table 1 were used. The samples were prepared by diluting the recombinant proteins with a TEST solution (20 mM Tris-HCl (pH 7.4), 400 mM NaCl, 0.1% Tween 20).

Each of the standard samples was added to wells (100 μL/well) and was then allowed to stand at room temperature for 1 hour. Then, the wells were washed with the TEST solution six times, and then a labeled detection antibody solution was added to the wells in an amount of 100 μL/well and allowed to stand at room temperature for 1 hour. The concentration of the labeled detection antibody used is shown in Table 1. The detection of galectin was performed in accordance with the protocol of a kit including the labeling protein. More specifically, the absorbance of the galectin-1 ELISA measurement system was measured at 595 nm, and the absorbance of the galectin-3 ELISA measurement system and the absorbance of the galectin-4 measurement system were measured at 450 nm. As an instrument for measuring absorbance, Tecan GENios (Tecan Group Ltd., Zurich, Switzerland) was used.

Table 2(a) shows the results of a spiked recovery test performed by adding a known amount of each of the recombinant proteins to plasma samples. As shown in Table 2(a), all the ELISA measurement systems examined in this example achieved recovery rates within the range of 84.4 to 108%. From the results, it has been confirmed that these ELISA measurement systems have no problems.

Table 2(b) shows the ratios (%) of measured values obtained by measuring known amounts of the recombinant proteins with the ELISA measurement systems to theoretical values (known concentrations). As shown in Table 2(b), it has been confirmed that as a result of the cross-reactivity test performed on the galectin ELISA measurement systems with the use of the standard samples (galectin-1, -2, -3, -4, and -7 recombinant proteins), each of the ELISA measurement systems has no reactivity with galectins other than its target galectin.

TABLE 2
(a) Spiked Recovery Rate

| | Galectin-1 | Galectin-2 | Galectin-3 | Galectin-4 | Galectin-7 |
|---|---|---|---|---|---|
| Recovery rate | 108% | 97.1% | 89.8% | 86.3% | 84.4% |

(b) Cross-Reactivity

| | Recombinant | Recombinant | Recombinant | Recombinant | Recombinant |
|---|---|---|---|---|---|
| | Human | Human | Human | Human | Human |
| | Galectin-1 | Galectin-2 | Galectin-3 | Galectin-4 | Galectin-7 |
| Galectin-1 ELISA | | 0% | 0% | 0% | 0% |
| Galectin-2 ELISA | 1% | | 0% | 0% | 0% |
| Galectin-3 ELISA | 0% | 0% | | 0% | 0% |
| Galectin-4 ELISA | 1% | 0% | 0% | | 0% |
| Galectin-7 ELISA | 0% | 0% | 0% | 0% | |

### <Conditions for Measurement of Collected Blood Sample>

As a measurement sample (i.e., a blood sample used to measure a galectin concentration), a plasma sample diluted with a TBST solution (20 mM Tris-HCl (pH 7.4), 400 mM NaCl, 0.1% Tween 20) was used. The dilution factor is shown in Table 1.

The measurement sample was added to wells (100 μL/well), and was then allowed to stand at room temperature for 1 hour. Then, the wells were washed with the TEST solution six times, and then a labeled detection antibody solution was added to the wells in an amount of 100 μL/well and allowed to stand at room temperature for 1 hour. The concentration of the labeled detection antibody used is shown in Table 1. The detection of galectin was performed in accordance with the protocol of a kit including the labeling protein. More specifically, the absorbance of the galectin-1 ELISA measurement system was measured at 595 nm, and the absorbance of the galectin-3 ELISA measurement system and the absorbance of the galectin-4 ELISA measurement system were measured at 450 nm. As an instrument for measuring absorbance, Tecan GENios (Tecan Group Ltd., Zurich, Switzerland) was used.

### Example 2

Blood samples (hereinafter, referred to as “plasma samples”) collected from patients who gave informed consent in accordance with the ethical guidelines of the faculty of medicine of Osaka University were analyzed in the following manner. The plasma samples were prepared in accordance with Reference Example 1 from blood collected from 105 colorectal cancer patients and 100 healthy individuals. Table 3 shows clinical information about the plasma samples used in this analysis. In this example, those whose levels of existing markers (more specifically, CEA, CA19-9, SCC antigen, CA125, CA15-3, and PSA) were all within normal limits were defined as “healthy individuals”.

TABLE 3

| | |
|---|---|
| Colorectal cancer | |
| patients (CRC) | Healty individuals (control) |

Age

| | | |
|---|---|---|
| Average(range, standard | 63.5 (28-88, 11.3) | 61.2(40-86, 9.9) |
| deviation) | | |
| Number of samples | 105 | 100 |

Sex

| | | |
|---|---|---|
| Man | 63 | 60 |
| Woman | 42 | 40 |

Disease state

| | |
|---|---|
| TNM classification | |
| Stage 0 | 6 |
| Stage I | 28 |
| Stage II | 25 |
| Stage III | 27 |
| Stage IV | 19 |

Cure (Curability)

| | |
|---|---|
| Cure A | 86 |
| Cure B | 8 |
| Cure C | 11 |

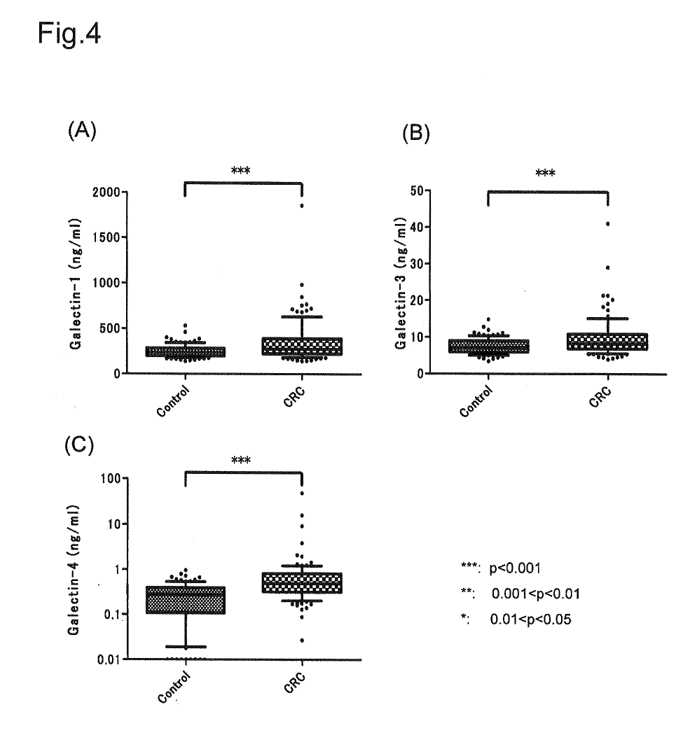

The blood samples of the healthy individuals and the cancer patients were analyzed to determine their galectin concentration and a comparison of the concentration of galectin in the blood samples was made between a group of the healthy individuals and a group of the cancer patients. FIG. 4(A) shows the results of galectin-1, FIG. 4(B) shows the results of galectin-3, and FIG. 4(C) shows the results of galectin-4. In each of the graphs, the vertical axis represents the concentration of galectin in the plasma samples. In each box plot, a box represents the range from 25th to 75th percentile of concentration distribution of all the samples, horizontal lines represent the range from 10th to 90th percentile of concentration distribution of all the samples, and a horizontal line in the box represents a median concentration in each group [Control (healthy individuals) or CRC (colorectal cancer patients)].

As shown in FIG. 4, there were statistically significant differences in the concentrations of all galectin-1, galectin-3, and galectin-4 between the two groups (Mann-Whitney test: p-value <0.001). The results indicate that galectin-1, galectin-3, and galectin-4 are useful as colorectal cancer clinical markers.

### Example 3

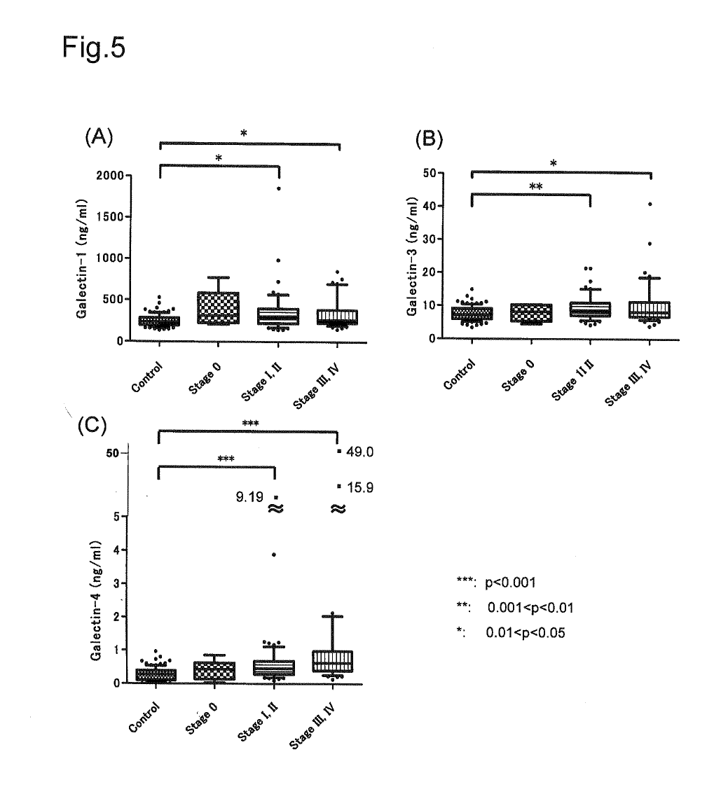

The 105 colorectal cancer patients were classified into 3 groups (Stage 0, Stage I-II, and Stage III-VI) according to TMN classification, and a comparison of galectin concentration was made among these groups. FIG. 5(A) shows the results of galectin-1, FIG. 5(B) shows the results of galectin-3, and FIG. 5(C) shows the results of galectin-4. In each of the graphs, the vertical axis represents the concentration of galectin in the plasma samples. In each box plot, a box represents the range from 25th to 75th percentile of concentration distribution of all the samples, horizontal lines represent the range from 10th to 90th percentile of concentration distribution of all the samples, and a horizontal line in the box represents a median concentration in each group [Control (healthy individuals) or CRC (colorectal cancer patients)].

As shown in FIG. 5, the galectin concentration was statistically significantly higher in the Stage I-II group and the Stage III-IV group than in the healthy individual group (non-parametric Kruskall-Wallis with Dunn's post test: p-value <0.05).

Further, galectin-4 showed a tendency that concentration thereof in the collected blood samples was higher in a more advanced cancer stage (FIG. 5(C)). On the other hand, galectin-1 and galectin-3 did not show such a tendency (FIGS. 5(A) and 5(B)). The results indicate that galectin-4 has features as a tumor progression marker.

### Example 4

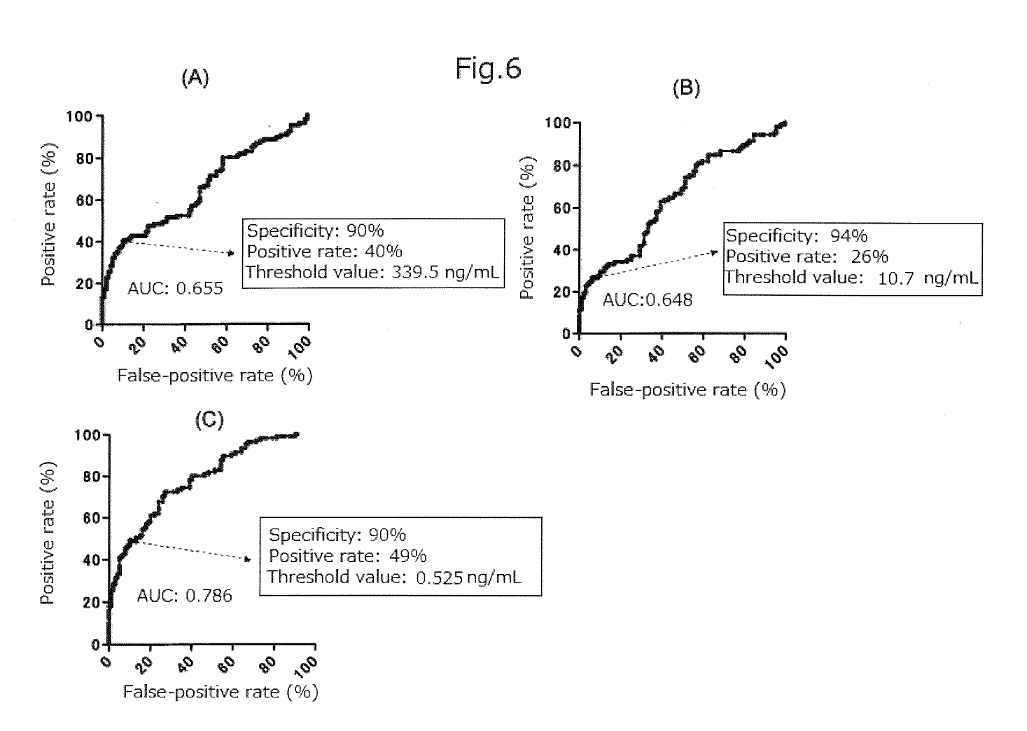

ROC (receiver operating characteristic) curves showing the discrimination between colorectal cancer patients and healthy individuals were generated based on the obtained respective galectin concentrations. FIG. 6(A) shows the ROC curve for galectin-1, FIG. 6(B) shows the ROC curve for galectin-3, and FIG. 6(C) shows the ROC curve for galectin-4. In FIG. 6, the vertical axis represents a positive rate and the horizontal axis represents a false-positive rate. The threshold value of galectin was determined by Youden's Index based on the ROC curve. More specifically, the threshold value of galectin-1 was set to 339.5 ng/mL, the threshold value of galectin-3 was set to 10.7 ng/mL, and the threshold value of galectin-4 was set to 0.525 ng/mL. Hereinbelow, detailed analysis was performed using the threshold value.

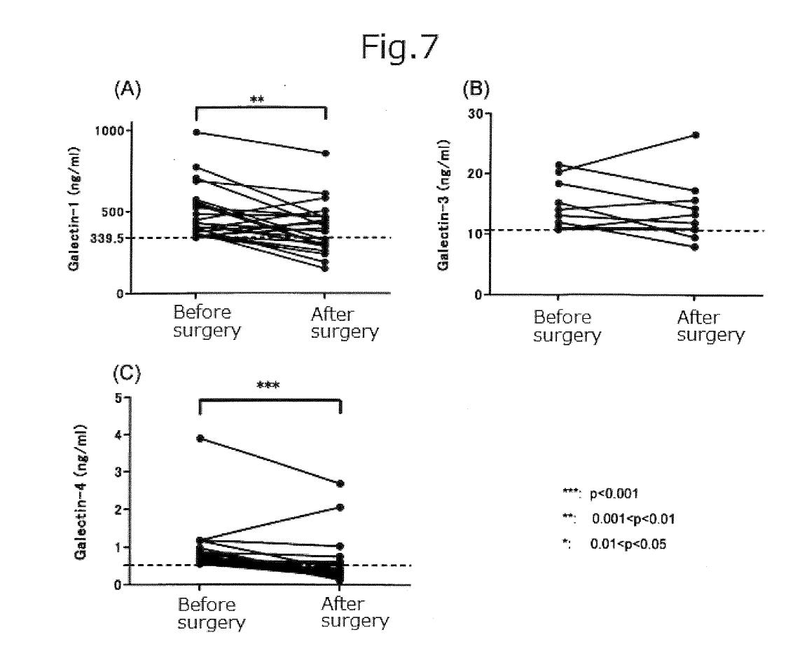

A comparison of the concentration of galectin in individuals positive for galectin concentration (i.e., in individuals whose galectin concentration exceeded the threshold value) (except for individuals with curability C) was made between before and after surgery. FIG. 7(A) shows the results of galectin-1, FIG. 7(B) shows the results of galectin-3, and FIG. 7(C) shows the results of galectin-4. In FIG. 7, plots connected by a line represent the concentrations of galectin in the same individual before and after surgery, and a broken line represents the threshold value determined by the ROC curve. As shown in FIG. 7, the concentrations of galectin-1 and galectin-4 in the collected blood samples were significantly reduced (Wilcoxon matched pairs test: p-value <0.01) after surgery (FIG. 7(A) and FIG. 7(C)).

The above results indicate that galectin-1 and galectin-4 are useful as follow-up markers.

### Example 5

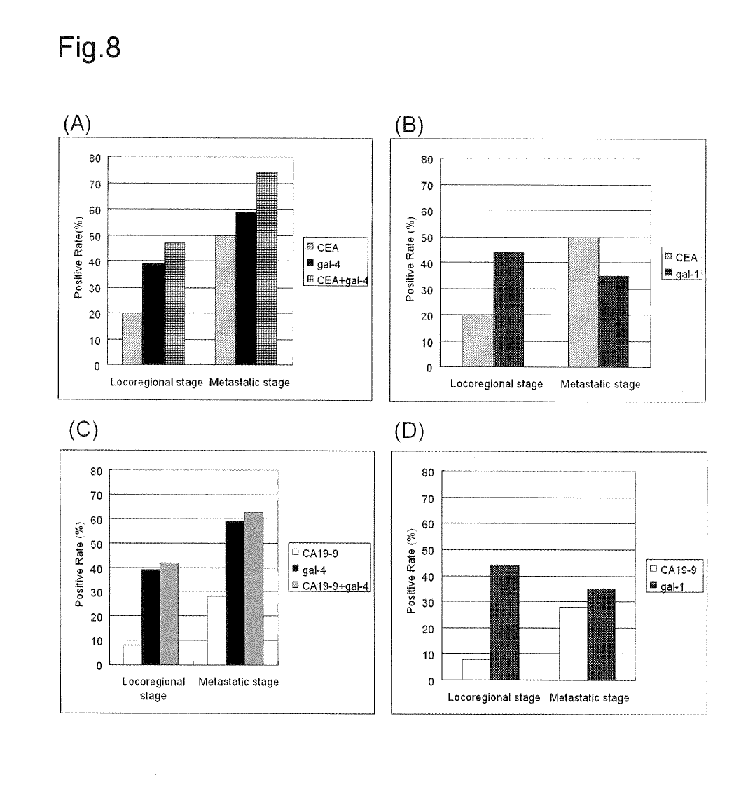

A comparison of patient capture rates (i.e., positive rates) of cancer patient groups in different disease states was made between when galectin-1 or galectin-4 was used as a marker and when only CEA or CA19-9 was used as a marker. FIG. 8(A) shows the results of comparison between CEA and galectin-4, FIG. 8(B) shows the results of comparison between CEA and galectin-1, FIG. 8(C) shows the results of comparison between CA19-9 and galectin-4, and FIG. 8(D) shows the results of comparison between CA19-9 and galectin-1.

As shown in FIG. 8, the positive rate when galectin-4 was used as a marker was higher in the patient group in an advanced metastatic stage (FIGS. 8(A) and 8(C)). On the other hand, it has been found that the positive rate when galectin-1 was used as a marker was relatively low in the patient group in a metastatic stage that is a relatively advanced stage, but, was higher in the patient group in a locoregional stage that is a relatively early stage (FIGS. 8(B) and 8(D)). It is to be noted that FIG. 8(A) shows also the positive rate when CEA and Galectin-4 were used in combination and FIG. 8(C) shows also the positive rate when CA19-9 and Galectin-4 were used in combination (in both cases, a case where at least one of the marker levels exceeded the threshold value was regarded as a positive case).

From the results, it has been found that galectin-1 has features as a tumor screening marker, and galectin-4 has features as a tumor progression marker.

### Example 6

The results of analysis performed using combinations of galectin-4 and an existing tumor progression marker, CEA and/or CA19-9 are shown in Table 4. As shown in Table 4, a patient capture rate (positive rate) was sufficiently improved when CEA or CA19-9 was used in combination with galectin-4 as compared to when only CEA or CA19-9 was used. This indicates that galectin-4 is useful as a tumor progression marker that complements the existing tumor progression marker.

TABLE 4

| | Positive rate |
|---|---|
| CEA | 33% |
| CA19-9 | 17% |
| Galectin-4 | 49% |
| CEA + CA19-9 | 36% |
| CEA + Galectin-4 | 59% |
| CA19-9 + Galectin-4 | 53% |
| CEA + CA19-9 + Galectin-4 | 60% |

1. A colorectal cancer marker selected from the group consisting of galectin-3 and galectin-4.

2. The colorectal cancer marker according to claim 1, wherein the galectin-4 is used as a tumor progression marker, a tumor screening marker, or a prognostic prediction marker.

3. The colorectal cancer marker according to claim 1, wherein the galectin-3 is used as a tumor screening marker.

4. A method of analyzing a galectin concentration in a collected blood sample, the method comprising the step Pₙ of measuring a concentration of a colorectal cancer marker selected from the group consisting of galectin-3 and galectin-4 in a collected blood sample Sₙ derived from an individual to acquire a measured value Cₙ[G3/G4] and comparing the measured value Cₙ[G3/G4] with a reference value C_{ref}[G3/G4] of the colorectal cancer marker, thereby analyzing the galectin concentration.

5. The method according to claim 4, further comprising, prior to the step Pₙ (n≧1), the step Pₙ₋₁ of measuring a concentration of galectin-4 in a collected blood sample Sₙ₋₁ derived from the same individual and collected before collection of the blood sample Sₙ to acquire a measured value Cₙ₋₁[G4], wherein
the reference value C_{ref}[G4] compared with the measured value Cₙ[G4] in the step Pₙ is selected from the group consisting of the measured value Cₙ₋₁[G4] and a threshold value Cₜₕ[G4] of galectin-4.

6. The method according to claim 5, comprising, prior to the step Pₙ (n≧2), the step P₁ of measuring a concentration of galectin-4 in a collected blood sample S₁ derived from the same individual and collected before collection of the blood sample Sₙ to acquire a measured value C₁[G4], and the step P₀ of measuring a concentration of galectin-4 in a collected blood sample S₀ derived from the same individual and collected before collection of the blood sample S₁ to acquire a measured value C₀[G4], wherein
the individual has undergone surgery for colorectal cancer between the step P₀ and the step P₁, the measured value C₀[G4] acquired in the step P₀ exceeds the threshold value Cₜₕ[G4] of galectin-4, and the measured value C₁[G4] acquired in the step P₁ is below the threshold value Cₜₕ[G4], and
the reference value C_{ref}[G4] compared with the measured value Cₙ[G4] in the step Pₙ is the threshold value Cₜₕ[G4].

7. The method according to claim 6, wherein the individual has further undergone non-surgical therapy for colorectal cancer between the step P₁ and the step Pₙ.

8. The method according to claim 5, wherein the individual has undergone at least non-surgical therapy for colorectal cancer between the step Pₙ₋₁ and the step Pₙ,
the measured value Cₙ₋₁[G4] acquired in the step Pₙ₋₁ exceeds the threshold value Cₜₕ[G4] of galectin-4, and the reference value C_{ref}[G4] compared with the measured value Cₙ[G4] in the step Pₙ is the threshold value Cₜₕ[G4] and the measured value Cₙ₋₁[G4].

9. The method according to claim 5, comprising, prior to the step Pₙ (n≧2), the step Pₙ₋₁ of measuring a concentration of galectin-4 in a collected blood sample Sₙ₋₁ derived from the same individual and collected before collection of the blood sample Sₙ to acquire a measured value Cₙ₋₁[G4], and the step P₀ of measuring a concentration of galectin-4 in a collected blood sample S₀ derived from the same individual and collected before collection of the blood sample Sₙ₋₁ to acquire a measured value C₀[G4], wherein
the individual has undergone at least non-surgical therapy for colorectal cancer between the step P₀ and the step Pₙ₋₁, and has subsequently undergone the non-surgical therapy also between the step Pₙ₋₁ and the step Pₙ, and wherein
the measured value C₀[G4] acquired in the step P₀ exceeds the threshold value Cₜₕ[G4] of galectin-4, and the reference value C_{ref}[G4] compared with the measured value Cₙ[G4] in the step Pₙ is the threshold value Cₜₕ[G4] and the measured value Cₙ₋₁[G4].

10. The method according to claim 4, wherein the reference value C_{ref}[G3/G4] of the colorectal cancer marker is a threshold value Cₜₕ[G3/G4] of the colorectal cancer marker.

11. Galectin-1 used as a tumor screening marker or a tumor progression marker for colorectal cancer.

12. A method of analyzing a galectin concentration in a collected blood sample, the method comprising the step Pₙ of measuring a concentration of galectin-1 in a collected blood sample Sₙ derived from an individual to acquire a measured value Cₙ[G1] and comparing the measured value Cₙ[G1] with a reference value C_{ref}[G1] of galectin-1, thereby analyzing the galectin concentration.

13. The method of analyzing a galectin concentration according to claim 12, wherein the reference value C_{ref}[G1] of galectin-1 is a threshold value Cₜₕ[G1] of galectin-1.

14. The method according to claim 12, further comprising, prior to the step Pₙ (n≧1), the step Pₙ₋₁ of measuring a concentration of galectin-1 in a collected blood sample Sₙ₋₁ derived from the same individual and collected before collection of the blood sample Sₙ to acquire a measured value Cₙ₋₁[G1], wherein
the reference value C_{ref}[G1] compared with the measured value Cₙ[G1] in the step Pₙ is selected from the group consisting of the measured value Cₙ₋₁[G1] and a threshold value Cₜₕ[G1] of galectin-1.

15. The method according to claim 14, comprising, prior to the step Pₙ (n≧2), the step P₁ of measuring a concentration of galectin-1 in a collected blood sample S₁ derived from the same individual and collected before collection of the blood sample Sₙ to acquire a measured value C₁[G1], and the step P₀ of measuring a concentration of galectin-1 in a collected blood sample S₀ derived from the same individual and collected before collection of the blood sample S₁ to acquire a measured value C₀[G1], wherein
the individual has undergone treatment for colorectal cancer with surgery between the step P₀ and the step P₁,
the measured value C₀[G1] acquired in the step P₀ exceeds the threshold value Cₜₕ[G1] of galectin-1, and the measured value C₁[G1] acquired in the step P₁ is below the threshold value Cₜₕ[G1], and
the reference value C_{ref}[G1] compared with the measured value Cₙ[G1] in the step Pₙ is the threshold value Cₜₕ[G1].

16. The method according to claim 15, wherein the individual has further undergone non-surgical therapy for colorectal cancer between the step P₁ and the step Pₙ.

17. The method according to claim 14, wherein the individual has undergone at least non-surgical therapy for colorectal cancer between the step Pₙ₋₁ and the step Pₙ,
the measured value Cₙ₋₁[G1] acquired in the step Pₙ₋₁ exceeds the threshold value Cₜₕ[G1] of galectin-1, and the reference value C_{ref}[G1] compared with the measured value Cₙ[G1] in the step Pₙ is the threshold value Cₜₕ[G1] and the measured value Cₙ₋₁[G1].

18. The method according to claim 14, comprising, prior to the step Pₙ (n≧2), the step Pₙ₋₁ of measuring a concentration of galectin-1 in a collected blood sample Sₙ₋₁ derived from the same individual and collected before collection of the blood sample Sₙ to acquire a measured value Cₙ₋₁[G1], and the step P₀ of measuring a concentration of galectin-1 in a collected blood sample S₀ derived from the same individual and collected before collection of the blood sample Sₙ₋₁ to acquire a measured value C₀[G1], wherein
the individual has undergone at least non-surgical therapy for colorectal cancer between the step P₀ and the step Pₙ₋₁, and has subsequently undergone the non-surgical therapy also between the step Pₙ₋₁ and the step Pₙ,
the measured value C₀[G1] acquired in the step P₀ exceeds a threshold value Cₜₕ[G1] of galectin-1, and the reference value C_{ref}[G1] compared with the measured value Cₙ[G1] in the step Pₙ is the threshold value Cₜₕ[G1] and the measured value Cₙ₋₁[G1].

19. The method according to claim 5, wherein as the threshold value, a concentration value of galectin-3 and/or a concentration value of galectin-4 that indicate(s) a specificity of 80% or higher is/are selected.

20. The method according to claim 5, wherein the step Pₙ further comprises analysis performed by measuring a concentration of another tumor progression marker for colorectal cancer in the collected blood sample Sₙ to acquire a measured value Cₙ[other] and comparing the measured value Cₙ[other] with a reference value C_{ref}[other] of the another tumor progression marker for colorectal cancer.

21. The method according to claim 20, wherein the another tumor progression marker for colorectal cancer is selected from the group consisting of carcinoembryonic antigen and CA19-9.

22. The method according to claim 4, wherein the measurement is performed by an immunoassay using a detection antibody selected from the group consisting of galectin-3 antibody and galectin-4 antibody that are labeled with a fluorescent compound and/or an enzyme protein.

23. The method according to claim 13, wherein as the threshold value, a concentration value of galectin-1 that indicates a specificity of 80% or higher is selected.

24. The method according to claim 14, wherein the step Pₙ further comprises analysis performed by measuring a concentration of another tumor progression marker for colorectal cancer in the collected blood sample Sₙ to acquire a measured value Cₙ[other] and comparing the measured value Cₙ[other] with a reference value C_{ref}[other] of the another tumor progression marker for colorectal cancer.

25. The method according to claim 24, wherein the another tumor progression marker for colorectal cancer is selected from the group consisting of carcinoembryonic antigen and CA19-9.

26. The method according to claim 12, wherein the measurement is performed by an immunoassay using, as a detection antibody, galectin-1 antibody labeled with a fluorescent compound and/or an enzyme protein.

27. A colorectal cancer marker detection kit comprising a detection antibody selected from the group consisting of galectin-1 antibody, galectin-3 antibody, and galectin-4 antibody that are labeled with a fluorescent compound and/or an enzyme protein.

28. The kit according to claim 27, wherein the enzyme protein is selected from the group consisting of peroxidase, alkaline phosphatase, and β-galactosidase.
